(19) United States
(12) Patent Application Publication
(10) Pub. No.: US 2008/0003213 A1
(43) Pub. Date: Jan. 3, 2008

(54) Methods and compositions for the treatment of diseases or conditions associated with increased C-reactive protein, interleukin-6, or interferon-gamma levels

(76) Inventor: Jan Lessem, Natick, MA (US)

Correspondence Address: CLARK & ELBING LLP, 101 FEDERAL STREET, BOSTON, MA, 02110, US

(21) Appl. No.: 11/804,916

(22) Filed: May 21, 2007

Related U.S. Application Data
(60) Provisional application No. 60/802,554, filed on May 22, 2006.

(51) Int. Cl. A61K 38/47; A61K 31/43; A61K 31/519; A61K 31/5377; A61K 31/5415; A61K 31/545; A61P 1/02; A61K 31/56; A61K 31/573; A61K 31/65; A61K 31/7036

(52) U.S. Cl. 424/94.61; 514/154; 514/171; 514/234.2; 514/262.1; 514/37

(57) ABSTRACT

The invention features methods and compositions for reducing the serum C-reactive protein (CRP), IL-6, and/or IFN-γ levels in a patient in need thereof, and for treating diseases and conditions associated with an increased serum CRP, IL-6, and/or IFN-γ levels. The invention also features methods and compositions for treating a patient diagnosed with, or at risk of developing, periodontal disease by administering a corticosteroid or an analog thereof and/or a tetra-substituted pyrimidopyrimidine or an adenosine analog upregulator.

### CROSS-REFERENCE TO RELATED APPLICATIONS

This application claims benefit from U.S. Provisional Application No. 60/802,554, filed May 22, 2006, hereby incorporated by reference.

### BACKGROUND OF THE INVENTION

The invention relates to the treatment of diseases and conditions associated with an increased serum C-reactive protein (CRP), interleukin-6 (IL-6), and/or interferon-γ (IFN-γ) levels.

CRP is an essential human acute-phase reactant produced in the liver in response to a variety of inflammatory cytokines. The protein is highly conserved and considered to be an early indicator of infectious or inflammatory conditions. Plasma CRP levels increase 1,000-fold in response to infection, ischemia, trauma, burns, and inflammatory conditions. Since the biological half-life of CRP is not influenced by age, liver or kidney function or pharmacotherapy, it is reliable biochemical marker for tissue destruction, necrosis and inflammation and its measurement is widely used to monitor various inflammatory states, angina pectoris, vascular insults, end-stage renal disease, rheumatoid arthritis, obesity and atherosclerosis.

CRP has long been used to monitor rheumatology, i.e., the activity of rheumatoid arthritis, and has recently been shown to be an independent marker for cardiovascular disease. The American Heart Association and Centers for Disease Control and Prevention issued statements recommending CRP be used as a risk marker for cardiovascular disease with a Framingham risk score of between 10% and 20%. Based on their recommendations, CRP levels <1 mg/L are considered low risk, levels from 1 to 3 mg/L represents average risk, and levels >3 mg/L are considered high risk.

IL-6 is a pro-inflammatory cytokine secreted by T cells and macrophages to stimulate immune response to trauma, especially burns or other tissue damage leading to inflammation. IL-6 has been strongly implicated in the genesis of autoimmune disorders, plasma cell neoplasias, inflammatory processes of the skin (including scleroderma, psoriasis and delayed pressure urticaria, rheumatoid arthritis juvenile chronic arthritis, coronary artery disease (CAD) with or without atherosclerosis, interstitial cystitis, and congestive heart failure.

IFN-γ is a lymphokine produced by activated T-lymphocytes and natural killer cells. It manifests anti-proliferative, antiviral and immunomodulatory activities and binds to a heterodimeric receptor on most primary cells of the immune system, and triggers a cascade of events leading to inflammation. The antiviral and immunomodulatory activity of IFN-γ is known to have beneficial effects in a number of clinical conditions. However, there are many clinical settings in which IFN-γ activity is known to have deleterious effects. For example, autoimmune diseases are associated with high levels of IFN.gamma. in the blood and diseased tissue from autoimmune patients. IFN-γ activity has also been linked to such disease states as cachexia and septic shock.

Agents that reduce the level of serum CRP, IL-6, and/or IFN-γ would be useful for treating numerous diseases and conditions.

### SUMMARY OF THE INVENTION

In one aspect, the invention features a method for treating periodontal disease (e.g., periodontitis, gingivitis) in a patient by administering (i) a corticosteroid; and (ii) a tetra-substituted pyrimidopyrimidine or an adenosine activity upregulator in amounts and for a duration that together are sufficient to treat periodontal disease.

In a related aspect, the invention features a method for reducing serum CRP, IL-6, and/or IFN-γ levels in a patient in need thereof by administering to the patient (i) a corticosteroid; and (ii) a tetra-substituted pyrimidopyrimidine or an adenosine activity upregulator in amounts and for a duration that together are sufficient to reduce serum CRP, IL-6, and/or IFN-γ levels in the patient. Desirably, the tetra-substituted pyrimidopyrimidine or adenosine activity upregulator may be administered in any useful dosage, e.g., 0.5-800 mg/day or 18-600 mg/day, in combination with a useful corticosteroid dosage, e.g., 0.1-1500 mg/day, 0.5-30 mg/day, or 0.5-10 mg/day. Compounds used in the methods of the invention may be formulated for, e.g., topical or systemic administration, and may be formulated in high, moderate, or low dosages.

In another related aspect, the invention features a method for treating a disease or condition associated with an increased serum CRP level (e.g., cardiovascular disease, atherosclerosis, hypertension, giant cell arteritis, Kawasaki disease, familial cold urticaria, angina pectoris, vascular insults, end-stage renal disease, rheumatoid arthritis, colon cancer, lymphoma, sarcoma, pancreatitis, or pancreatic cancer) in a patient in need thereof by administering to the patient (i) a corticosteroid; and (ii) a tetra-substituted pyrimidopyrimidine or an adenosine activity upregulator, wherein the two drugs are administered in amounts and for a duration that together are sufficient to reduce the serum CRP level in the patient.

In another related aspect, the invention features a method for treating a disease or condition associated with an increased IL-6 level (e.g., nephritis, mesangial proliferative nephritis, Crohn's disease, ulcerative colitis, pancreatitis, scleroderma, psoriasis, juvenile idiopathic arthritis or systemic juvenile idiopathic arthritis, vasculitis, Kawasaki disease, rheumatoid arthritis, systemic lupus erythematosus, psoriasis, Sjogren syndrome, adult Still's disease, acute transplant rejection, graft-versus-host disease, delayed pressure urticaria, osteoporosis, Castleman's disease, multiple myeloma, diabetes, cachexia, interstitial pneumonia, bronchial asthma, vasculitis syndrome, cardiac mixoma, Kaposi's sarcoma, Lyme disease, coronary artery disease (CAD) with or without atherosclerosis, interstitial cystitis, congestive heart failure, multiple sclerosis, organ failure following burn, septic shock, Paget's disease, myeloma bone disease, hypercholesterolemia, lymphopenia, cancer, liver cirrhosis/fibrosis, scar formation, influenza, tuberculosis, or cholera) in a patient in need thereof by administering to the patient (i) a corticosteroid; and (ii) a tetra-substituted pyrimidopyrimidine or an adenosine activity upregulator, wherein the two drugs are administered in amounts and for a duration that together are sufficient to reduce the IL-6 level in the patient.

In another related aspect, the invention features a method for treating a disease or condition associated with an increased IFN-γ level (e.g., ovarian cancer, alveolar echinococcosis, Lyme disease, fungal liver abscess, mycobacterial infection, vaccine-associated bacille Calmette Guerin, salmonella, hepatitis, Brucella abortus infection, Whipples disease, enteritis, suppurative lymphadenitis, pneumonia, Aspergillus infection, abscesses of the lung, liver, or spleen, septic shock/cachexia, arteriosclerosis, suppression of bone resoprtion, hypercatabolic states (e.g., burn trauma), multiple sclerosis, idiopathic pulmonary fibrosis, chronic granulomatous disease, graft versus host disease, orceliac disease) in a patient in need thereof by administering to the patient (i) a corticosteroid; and (ii) a tetra-substituted pyrimidopyrimidine or an adenosine activity upregulator, wherein the two drugs are administered in amounts and for a duration that together are sufficient to reduce the IFN-γ level in the patient.

Cancers treated according to any of the methods of the invention can be, for example, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, and retinoblastoma. Preferably, the cancer being treated is lung cancer, especially lung cancer attributed to squamous cell carcinoma, adenocarinoma, or large cell carcinoma, colorectal cancer, ovarian cancer, especially ovarian adenocarcinoma, prostate cancer; gastric cancer, esophageal cancer, head and neck cancer, or thyroid cancer.

In any of the foregoing aspects, the two drugs can be formulated in a single pharmaceutical composition, or can be in separate formulations and administered simultaneously (i.e., within an hour of each other), within 2, 4, 6, 8, 12, or 16 hours of each other, or within 1, 5, 7, 10, or 14 days of each other.

In any of the above aspects, a third agent may be optionally administered to the patient. Suitable agents include antibiotics (penicillin, cephalosporin, tetracycline, oxytetracycline, chlortetracycline, metronidazole, chloramphenicol, streptomycin, neomycin, sulfonamides, phenolic compounds, quartemary ammonium compounds, minocycline, doxycycline); antiseptics (e.g., chlorhexidine); NSAIDs (e.g., flurbiprofen, carprofen, diclofenac, fenbufen, fenclozic acid, fenoprofen, flufenamic acid, ibuprofen, indomethacin, indoprofen, ketoprofen, lonazolac, loxoprofen, meclofenamic acid, mefanamic acid, naproxen, proprionic acids, salicylic acids, sulindac, tolmetin, meloxicam, oxicams, piroxicam, tenoxicam, etodolac, and oxaprozin); tranexamic acid, allantoin; epsilon-aminocaproic acid; lysozyme; dihydrocholesterol; beta-glycyrrhetinic acid; platelet aggregation inhibitors (e.g., abciximab, aspirin, cilostazol, clopidogrel, eptifibatide, ticlopidine, or tirofiban); anticoagulants (e.g., dalteparin, danaparoid, enoxaparin, heparin, tinzaparin, or warfarin); antipyretics (e.g., acetaminophen); ticlopidine; clopidogrel; angiotensin converting enzyme inhibitors; beta blockers; pentoxifylline; cilostazol; estrogen replacement therapy; and lipid-lowering agents (e.g., cholestyramine, colestipol, nicotinic acid, gemfibrozil, probucol, ezetimibe, or statins such as atorvastatin, rosuvastatin, lovastatin simvastatin, pravastatin, cerivastatin, and fluvastatin). These secondary therapeutic agents may be administered within 14 days, 7 days, 1 day, or 12 hours of administration of a corticosteroid and/or a tetra-substituted pyrimidopyrimidine, or simultaneously therewith. The additional therapeutic agents may be present in the same or different pharmaceutical compositions as the corticosteroid and/or tetra-substituted pyrimidopyrimidine of the invention. When present in different pharmaceutical compositions, different routes of administration may be used.

In other embodiments, the corticosteroid and the tetra-substituted pyrimidopyrimidine are the only two active ingredients (although excipients will generally also be present).

The invention also features a device for administering drugs to the periodontal pockets of a patient having periodontal disease. The device includes a corticosteroid and a tetra-substituted pyrimidopyrimidine or an adenosine activity upregulator capable of being released into the periodontal pockets of the patient in periodontal disease-treating amounts. Additional drugs, such as those listed above, can also be included in this device.

The invention also features a variety of kits. One kit includes (i) a corticosteroid; (ii) a tetra-substituted pyrimidopyrimidine or an adenosine activity upregulator; and (iii) instructions for administering drugs to a patient having or at risk of having periodontal disease, a patient having increased serum CRP, IL-6, and/or IFN-γ levels, or a patient having or at risk of having periodontal disease. In one embodiment, the two drugs are contained within a single composition.

Another kit of the invention includes either a corticosteroid or a tetra-substituted pyrimidopyrimidine or an adenosine activity upregulator and instructions for administering both a corticosteroid and a tetra-substituted pyrimidopyrimidine or an adenosine activity upregulator to a patient having periodontal disease, a patient having increased serum CRP, IL-6, and/or IFN-γ levels, or a patient having or at risk of having periodontal disease.

In certain embodiments of any of the foregoing aspects of the invention, the corticosteroid is prednisolone or prednisone, and the tetra-substituted pyrimidopyrimidine is dipyridamole.

While the invention is described generally in terms of combination therapy, it is understood that either agent (i.e., the corticosteroid or the tetra-substituted pyrimidopyrimidine or an adenosine activity upregulator) can be used as a monotherapy to treat periodontal disease or reduce the serum CRP level in a patient needing such treatment. Thus, any of the foregoing methods can be performed (and any of the foregoing devices produced) employing only a corticosteroid or only a tetra-substituted pyrimidopyrimidine. As an example, in one embodiment, the invention features a method of treating periodontal disease by administering dipyridamole as a monotherapy to treat periodontal disease or reduce serum CRP, IL-6, and/or IFN-γ levels.

In any of the foregoing aspects, the corticosteroid can be replaced with a non-steroidal immunophilin-dependent immunosuppressant, small molecule immunomodulator, glucocorticoid receptor modulator, or NSAID, as is described in greater detail below.

By “corticosteroid” is meant any naturally occurring or synthetic compound characterized by a hydrogenated cyclopentanoperhydro-phenanthrene ring system and having immunosuppressive and/or antinflammatory activity. Naturally occurring corticosteriods are generally produced by the adrenal cortex. Synthetic corticosteroids may be halogenated. Examples of corticosteroids are provided herein.

By a “dosage equivalent to a prednisolone dosage” is meant a dosage of a corticosteroid that, in combination with a given dosage of a tetra-substituted pyrimidopyrimidine produces the same anti-inflammatory effect in a patient as a dosage of prednisolone in combination with that dosage.

By “non-steroidal immunophilin-dependent immunosuppressant” or “NsIDI” is meant any non-steroidal agent that decreases proinflammatory cytokine production or secretion, binds an immunophilin, or causes a down regulation of the proinflammatory reaction. NsIDIs include calcineurin inhibitors, such as cyclosporine, tacrolimus, ascomycin, pimecrolimus, as well as other agents (peptides, peptide fragments, chemically modified peptides, or peptide mimetics) that inhibit the phosphatase activity of calcineurin. NsIDIs also include rapamycin (sirolimus) and everolimus, which bind to an FK506-binding protein, FKBP-12, and block antigen-induced proliferation of white blood cells and cytokine secretion.

By “small molecule immunomodulator” is meant a non-steroidal, non-NsIDI compound that decreases proinflammatory cytokine production or secretion, causes a down regulation of the proinflammatory reaction, or otherwise modulates the immune system in an immunophilin-independent manner. Examplary small molecule immunomodulators are p38 MAP kinase inhibitors such as VX 702 (Vertex Pharmaceuticals), SCIO 469 (Scios), doramapimod (Boehringer Ingelheim), RO 30201195 (Roche), and SCIO 323 (Scios), TACE inhibitors such as DPC 333 (Bristol Myers Squibb), ICE inhibitors such as pranalcasan (Vertex Pharmaceuticals), and IMPDH inhibitors such as mycophenolate (Roche) and merimepodib (Vertex Pharamceuticals).

By “tetra-substituted pyrimidopyrimidine” is meant a compound of formula (V): 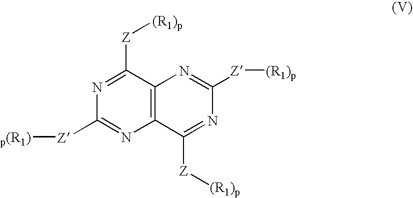
wherein each Z and each Z′ is, independently, N, O, C,

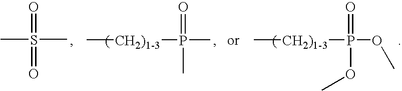

When Z or Z′ is O or 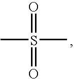
then p=1, when Z or Z′ is N, 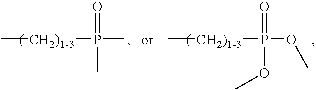
then p=2, and when Z or Z′ is C, then p=3. In formula (V), each R₁ is, independently, X, OH, N-alkyl (wherein the alkyl group has 1 to 20, more preferably 1-5, carbon atoms); a branched or unbranched alkyl group having 1 to 20, more preferably 1-5, carbon atoms; or a heterocycle, preferably as defined in formula (Y), below. Alternatively, when p>1, two R₁ groups from a common Z or Z′ atom, in combination with each other, may represent —(CY₂)ₖ— in which k is an integer between 4 and 6, inclusive. Each X is, independently, Y, CY₃, C(CY₃)₃, CY₂CY₃, (CY₂)₁₋₅OY, substituted or unsubstituted cycloalkane of the structure CₙY₂ₙ₋₁, wherein n=3-7, inclusive. Each Y is, independently, H, F, Cl, Br, or I. In one embodiment, each Z is the same moiety, each Z′ is the same moiety, and Z and Z′ are different moieties.

Particularly useful tetra-substituted pyrimidopyrimidines for use in the methods, kits, and compositions of the invention are dipyridamole (also known as 2,6-bis(diethanolamino)-4,8-dipiperidinopyrimido(5,4-d)pyrimidine); 2,6-disubstituted 4,8-dibenzylaminopyrimido[5,4-d]pyrimidines; mopidamole; dipyridamole monoacetate; R-E 244 (1-((2,7-bis(2-methyl-4-morpholinyl)-6-phenyl-4-pteridinyl)(2-hydroxyethyl)amino)-2-propanol); TX-3301 (asasantin); NU3026 (2,6-di-(2,2-dimethyl-1,3-dioxolan-4-yl)-methoxy-4,8-di-piperidinopyrimidopyrimidine); NU3059 (2,6-bis-(2,3-dimethyoxypropoxy)-4,8-di-piperidinopyrimidopyrimidine); NU3060 (2,6-bis[N,N-di(2-methoxy)ethyl]-4,6-di-piperidinopyrimidopyrimidine); and NU3076 (2,6-bis(diethanolamino)-4,8-di-4-methoxybenzylaminopyrimidopyrimidine). Other tetra-substituted pyrimidopyrimidines are described in U.S. Pat. Nos. 3,031,450 and 4,963,541, hereby incorporated by reference.

By “adenosine activity upregulator” is meant adenosine and any compounds that mimic or potentiate the physiological effects of adenosine, such as adenosine receptor agonists, adenosine transport inhibitors, adenosine kinase inhibitors, and phosphodiesterase (PDE) inhibitors, as described herein.

By a “low dosage” is meant at least 5% less (e.g., at least 10%, 20%, 50%, 80%, 90%, or even 95%) than the lowest standard recommended dosage of a particular compound formulated for a given route of administration for treatment of any human disease or condition. For example, a low dosage of tetra-substituted pyrimidopyrimidine formulated for administration by inhalation will differ from a low dosage of tetra-substituted pyrimidopyrimidine formulated for oral administration.

By a “high dosage” is meant at least 5% (e.g., at least 10%, 20%, 50%, 100%, 200%, or even 300%) more than the highest standard recommended dosage of a particular compound for treatment of any human disease or condition.

By a “moderate dosage” is meant the dosage between the low dosage and the high dosage.

By “treating” is meant administering or prescribing a pharmaceutical composition for the treatment or prevention of a disease or condition.

By “patient” is meant any animal (e.g., a human). Other animals that can be treated using the methods, compositions, and kits of the invention include horses, dogs, cats, pigs, goats, rabbits, hamsters, monkeys, guinea pigs, rats, mice, lizards, snakes, sheep, cattle, fish, and birds. In one embodiment of the invention, the patient subject to a treatment described herein does not have clinical depression, an anxiety or panic disorder, an obsessive/compulsive disorder, alcoholism, an eating disorder, an attention-deficit disorder, a borderline personality disorder, a sleep disorder, a headache, premenstrual syndrome, an irregular heartbeat, schizophrenia, Tourette's syndrome, or phobias.

By “an amount sufficient” is meant the amount of a compound, in a combination of the invention, required to treat or prevent a disease or condition in a clinically relevant manner. A sufficient amount of an active compound used to practice the present invention for therapeutic treatment of particular diseases and conditions caused varies depending upon the manner of administration, the age, body weight, and general health of the patient. Ultimately, the prescribers will decide the appropriate amount and dosage regimen.

By “more effective” is meant that a method, composition, or kit exhibits greater efficacy, is less toxic, safer, more convenient, better tolerated, or less expensive, or provides more treatment satisfaction than another method, composition, or kit with which it is being compared. Efficacy may be measured by a skilled practitioner using any standard method that is appropriate for a given indication.

The term “periodontal disease” encompasses a variety of conditions, including gingivitis and periodontitis, as well as diseases of tissues that surround and support teeth, including the gingiva, cementum, periodontal ligament, alveolar process bone, and dental supporting bone.

By “a disease or condition associated with an increased serum CRP level” is meant any disease or disorder in which the level of serum CRP may be elevated compared to normal controls. Typically a serum CRP level of >3 mg/L is considered elevated. Such diseases and conditions associated with an increased serum CRP level include cardiovascular disease (e.g., coronary artery disease, peripheral artery disease), hypertension, colon cancer, lymphoma, sarcoma, pancreatic cancer, and pancreatitis.

By “sustained release” or “controlled release” is meant that the therapeutically active component is released from the formulation at a controlled rate such that therapeutically beneficial blood levels (but below toxic levels) of the component are maintained over an extended period of time ranging from e.g., about 12 to about 24 hours, thus, providing, for example, a 12 hour or a 24 hour dosage form.

The term “pharmaceutically acceptable salt” represents those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. The salts can be prepared in situ during the final isolation and purification of the compounds of the invention, or separately by reacting the free base function with a suitable organic acid. Representative acid addition salts include acetate, adipate, alginate, ascorbate, aspartate, benzenesulfonate, benzoate, bisulfate, borate, butyrate, camphorate, camphersulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, isethionate, lactobionate, lactate, laurate, lauryl sulfate, malate, maleate, malonate, mesylate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

Compounds useful in the invention include those described herein in any of their pharmaceutically acceptable forms, including isomers such as diastereomers and enantiomers, salts, esters, amides, thioesters, solvates, and polymorphs thereof, as well as racemic mixtures and pure isomers of the compounds described herein. As an example, by “prednisolone” is meant the free base as well as any pharmaceutically acceptable salt thereof (e.g., prednisolone acetate).

Compounds useful in the invention may also be isotopically labeled compounds. Useful isotopes include hydrogen, carbon, nitrogen, oxygen, phosphorous, fluorine, and chlorine, (e.g., ²H, ³H, ¹³C, ¹⁴C, ¹⁵N, ¹⁸O, ¹⁷O, ³¹p, ³²P, ³⁵S, ¹⁸F, and ³⁶Cl). Isotopically-labeled compounds can be prepared by synthesizing a compound using a readily available isotopically-labeled reagent in place of a non-isotopically-labeled reagent.

In the generic descriptions of compounds of this invention, the number of atoms of a particular type in a substituent group is generally given as a range, e.g., an alkyl group containing from 1 to 7 carbon atoms or C₁₋₇ alkyl. Reference to such a range is intended to include specific references to groups having each of the integer number of atoms within the specified range. For example, an alkyl group from 1 to 7 carbon atoms includes each of C₁, C₂, C₃, C₄, C₅, C₆, and C₇. A C₁₋₇ heteroalkyl, for example, includes from 1 to 7 carbon atoms in addition to one or more heteroatoms. Other numbers of atoms and other types of atoms may be indicated in a similar manner.

Other features and advantages of the invention will be apparent from the following detailed description, and from the claims.

### DETAILED DESCRIPTION

The invention features methods and compositions for treating a patient diagnosed with, or at risk of developing, periodontal disease by administering a corticosteroid or an analog thereof and/or a tetra-substituted pyrimidopyrimidine or an analog thereof (e.g., an adenosine activity upregulator) to the patient. The invention also features methods and compositions for reducing serum CRP, IL-6, and/or IFN-γ levels in a patient in need thereof, and for treating diseases and conditions associated with increased serum CRP, IL-6, and/or IFN-γ levels.

In one embodiment of the invention, treatment of periodontal disease is performed by administering a corticosteroid and dipyridamole to a patient in need of such treatment.

The invention is described in greater detail below.

Tetra-Substituted Pyrimidopyrimidines

Tetra-substituted pyrimidopyrimidines that are useful in the methods, compositions, and kits of this invention include 2,6-disubstituted 4,8-dibenzylaminopyrimido[5,4-d]pyrimidines. Particularly useful tetra-substituted pyrimidopyrimidines include dipyridamole (also known as 2,6-bis(diethanolamino)-4,8-dipiperidinopyrimido(5,4-d)pyrimidine); mopidamole; dipyridamole monoacetate; R-E 244 (1-((2,7-bis(2-methyl-4-morpholinyl)-6-phenyl-4-pteridinyl)(2-hydroxyethyl)amino)-2-propanol); TX-3301 (asasantin); NU3026 (2,6-di-(2,2-dimethyl-1,3-dioxolan-4-yl)-methoxy-4,8-di-piperidinopyrimidopyrimidine); NU3059 (2,6-bis-(2,3-dimethyoxypropoxy)-4,8-di-piperidinopyrimidopyrimidine); NU3060 (2,6-bis[N,N-di(2-methoxy)ethyl]-4,6-di-piperidinopyrimidopyrimidine); and NU3076 (2,6-bis(diethanolamino)-4,8-di-4-methoxybenzylaminopyrimidopyrimidine). Other tetra-substituted pyrimidopyrimidines are described in U.S. Pat. Nos. 3,031,450 and 4,963,541.

The standard recommended dosage for dipyridamole is 300-400 mg/day.

Adenosine and Adenosine Activity Upregulators

Dipyridamole is an adenosine activity upregulator. If desired, another adenosine activity upregulator can be used in place of dipyridamole in the methods, compositions, and kits of the invention. Suitable adenosine activity upregulators are adenosine receptor agonists, adenosine transport inhibitors, adenosine kinase inhibitors, and phosphodiesterase (PDE) inhibitors, discussed below.

Adenosine Receptor Agonists

Examples of adenosine receptor agonists that can be employed in the methods, compositions, and kits of the invention are adenosine hemisulfate salt, adenosine amine congener solid, N⁶-(4-amino-3-iodophenyl)methyl-5′-N-methylcarboxamidoadenosine (I-AB-MECA); N-((2-methylphenyl)methyl)adenosine (Metrifudil); 2-(1-hexynyl)-N-methyladenosine (HEMADO); N-(1-methyl-2-phenylethyl)adenosine (R-PIA); N⁶—(R-4-hydroxyphenylisopropyl)adenosine (HPIA); N⁶-cyclopentyladenosine (CPA); N⁶-cyclopentyl-2-(3-phenylaminocarbonyltriazene-1-yl)adenosine (TCPA); N-((1S, trans)-2-hydroxycyclopentyl)adenosine (GR 79236); N⁶-cyclohexyladenosine (CHA); 2-chloro-N⁶-cyclopentyladenosine (CCPA); N-ethylcarboxamidoadenosine (NECA); 2-(4-(2-carboxyethyl)phenethylamino)-5′-N-ethylcarboxamidoadenosine (CGS 21680); N⁶-(3-iodobenzyl)-5′-N-methylcarboxamidoadenosine (IB-MECA); 2-(cyclohexylmethylidene hydrazino)adenosine (WRC 0470); 2-(4-(2-carboxyethyl)phenethylamino)-5′-N-ethylcarboxamidoadenosine (CGS 21680); N⁶-(2-(3,5-dimethoxyphenyl)-2-(2-methylphenyl)ethyl)adenosine (DPMA); hexynyladenosine-5′-N-ethylcarboxamide (HE-NECA); 2-[(2-aminoethyl-aminocarbonylethyl)phenylethylamino]-5′-N-ethyl-carboxamidoadenosine (APEC); 2-chloro-N⁶-(3-iodobenzyl)-5′-N-methylcarboxamidoadenosine (2-Cl-IB-MECA); 2-phenylaminoadenosine (CV 1808); 3′-Aminoadenosine-5′-uronamides; CV Therapeutics™ small molecule drugs Tecadenoson (CVT-510); Regadenoson (CVT 3146); and Carisa (CVT 3033); and Aderis Pharmaceuticals™ small drug molecules 2-[2-(4-chlorophenyl)ethoxy]adenosine (MRE 0094), 1-deoxy-1-[6-[[(iodophenyl)methyl]amino]-9H-purine-9-yl]-N-methyl-(-D-ribofuranuronamide) (CF101), Selodenoson (DTI-0009) and Binodenoson (MRE-0470). Other adenosine receptor agonists are those described or claimed in Gao et al., JPET, 298: 209-218 (2001); U.S. Pat. Nos. 5,278,150, 5,877,180, 6,232,297; U.S. Patent Application Publication No. 2005-0261236, and PCT Publication No. WO 98/08855, incorporated herein by reference.

Adenosine Transport Inhibitors

Adenosine transport inhibitors that can be employed in the methods, compositions, and kits of the invention include 3-[1-(6,7-diethoxy-2-morpholinoquinazolin-4-yl)piperidin-4-yl]-1,6-dimethyl-2,4(1H,3H)-quinazolinedione hydrochloride (KF24345); 6-(4-nitrobenzyl)-thioinosine (NBI) and 6-(2-hydroxy-5-nitrobenzyl)-thioguanosine (NBG); 6-[4-(1-cyclohexyl-1H-tetrazol-5-yl)butoxy]-3,4-dihydro-2(1H)-quinolinone (Cilostazol); (2-amino-4,5-dimethyl-3-thienyl)-[3-(trifluoromethyl) pheny]methanone (PD 81723); 3,7-dihydro-3-methyl-1-(5-oxohexyl)-7-propyl-1H-purine-2,6-dione(propentofylline); 6-[(4-nitrobenzyl)thio]-9-β-D-ribofuranosylpurine(nitrobenzylthioinosine) (NBMR); 3,4,5-trimethoxy-, (tetrahydro-1H-1,4-diazepine-1,4(5H)-diyl)di-3,1-propanediyl benzoic acid, ester (dilazep); hexobendine; dipyridamole; and adenosine transport inhibitors described in Fredholm, J. Neurochem. 62:563-573 (1994), Noji et al., J. Pharmacol. Exp. Ther. 300:200-205 (2002); and Crawley et al.; Neurosci. Lett. 36:169-174 (1983), each of which is incorporated herein by reference.

Adenosine Kinase Inhibitors

Adenosine kinase inhibitors are adenosine activity upregulators that can be used in the methods, compositions, and kits of the invention. Adenosine kinase inhibitors are generally described as either nucleoside-like, or nonnucleoside-like.

Nucleoside-Like Adenosine Kinase Inhibitors

Nucleoside-like adenosine kinase inhibitors that can be used in the methods, compositions, and kits of the invention include 5-iodotubercidin (5IT) and 2-diaryltubercidin analogues; 5′-deoxo-5′-deoxy-5-iodotubercidin (5′d-5IT); and 5′-deoxo-5′-aminoadenosine (NH₂dADO). Other nucleoside-like adenosine kinase inhibitors are described in McGaraughty et al., Current Topics in Medicinal Chemistry 5:43-58 (2005); Ugarkar, J. Med. Chem. 43:2883-2893 (2000); Ugarkar et al., J. Med. Chem. 43:2894-2905 (2000); Kaplan and Coyle, Eur. J. Pharmacol. 1:1-8 (1998); and Sinclair et al. Br. J. Pharmacol. 5:1037-1044 (2001), each of which is incorporated herein by reference.

Nonnucleoside-Like Adenosine Kinase Inhibitors

Nonnucleoside-like adenosine kinase inhibitors that can be used in the methods, compositions, and kits of the invention include 5-bromopyrrolopyrrolidine; 4-amino-5-(3-bromophenyl)-7-(6-morpholino-pyridin-3-yl)pyrido[2,3-d]pyrimidine (ABT-702). Other nonnucleoside-like AK inhibitors are described in McGaraughty et al., Current Topics in Medicinal Chemistry 5:43-58 (2005), Gomtsyan and Lee, Current Pharmaceutical Design 10:1093-1103 (2004); Jarvis et al. J. Pharm. Exp. Ther. 295:1156-1164 (2000); Kowaluk, et al. J. Pharm. Exp. Ther. 295:1165-1174 (2000); and German Patent Application DE 10141212 A1, each of which is incorporated herein by reference.

Phosphodiesterase Inhibitors

Several isozymes of phosphodiesterases act as regulatory switches by catalyzing the degradation of cAMP to adenosine-5-monophosphate (5′-AMP). Inhibitors of phosphodiesterases can lead to an increase in cAMP levels, which in turn can lead to an increase in antiinflammatory actions.

Type I Phosphodiesterase Inhibitors

Type I PDE inhibitors that can be employed in the methods, compositions, and kits of the invention include (3-alpha,16-alpha)-eburnamenine-14-carboxylic acid ethyl ester (Vinpocetine); 1 8-methoxymethyl-3-isobutyl-1-methylxantine (MIMX); 1-carboxy-2,3,4,4a,4b,5,6,6a,6b,7,8,8a,8b,9,10,10a,14,16,17,17a,17b,18,19,19a,19b, 20,21,21a,21b,22,23,23a-dotriacontahydro-14-hydroxy-8a,10a-bis(hydroxymethyl)-14-(3-methoxy-3-oxopropyl)-1,4,4a, 6,6a,17b,19b,21b-octamethyl beta-D-glucopyranosiduronic acid (Ks-505a); cis-5,6a,7,8,9,9a-hexahydro-2-(4-(trifluoromethyl)phenylmethyl)-5-methyl-cyclopent (4,5)imidazo(2,1-b)purin-4(3H)-one (SCH 51866); and 2-o-propoxyphenyl-8-azapurine-6-one (Zaprinast). Other Type I PDE inhibitors are described in U.S. Patent Application Publication Nos. 2004-0259792 and 2005-0075795, incorporated herein by reference.

Type II Phosphodiesterase Inhibitors

Type II PDE inhibitors that can be employed in the methods, compositions, and kits of the invention include erythro-9-(2-hydroxy-3-nonyl)adenine (EHNA); 2,3,6,7-tetrahydro-9,10-dimethoxy-3-methyl-2-((2,4,6-trimethylphenyl)imino)-4H-pyrimido(6,1-a)isoquinolin-4-one (trequinsin); ND7001 (Neuro3D Pharmaceuticals); and BAY 60-7550 (Alexis Biochemicals). Other Type II PDE inhibitors are described in U.S. Patent Application Publication No. 2003-0176316, incorporated herein by reference.

Type III Phosphodiesterase Inhibitors

Type III PDE inhibitors that can be employed in the methods, compositions, and kits of the invention include 3-isobutyl-1-methylxanthine (IBMX); 6-dihydro-2-methyl-6-oxo-3,4′-bipyridine)-5-carbonitrile (milrinone); and N-cyclohexyl-4-((1,2-dihydro-2-oxo-6-quinolinyl)oxy)-N-methyl-butanamide(cilostamide). Other Type III PDE inhibitors are described in the following patents and patent applications: EP 0 653 426, EP 0 294 647, EP 0 357 788, EP 0 220 044, EP 0 326 307, EP 0 207 500, EP 0 406 958, EP0 150 937, EP 0 075 463, EP 0 272 914, and EP 0 112 987, U.S. Pat. Nos. 4,963,561; 5,141,931, 6,897,229, and 6,156,753; U.S. Patent Application Publication Nos. 2003-0158133, 2004-0097593, 2006-0030611, and 2006-0025463; PCT Publication No. WO 96/15117; DE 2825048; DE 2727481; DE 2847621; DE 3044568; DE 2837161; and DE 3021792, each of which is incorporated herein by reference.

Type IV Phosphodiesterase Inhibitors

Type IV PDE inhibitors that can be employed in the methods, compositions, and kits of the invention include 4-(3-cyclopentyloxy-4-methoxyphenyl)-2-pyrrolidone(rolipram) and 4-(3-butoxy-4-methoxybenzyl)-2-imidazolidinone (Ro20-1724). Other Type IV PDE inhibitors are described in the following patents, patent applications, and references: U.S. Pat. Nos. 3,892,777, 4,193,926, 4,655,074, 4,965,271, 5,096,906, 5,124,455, 5,272,153, 6,569,890, 6,953,853, 6,933,296, 6,919,353, 6,953,810, 6,949,573, 6,909,002, and 6,740,655; U.S. Patent Application Publication Nos. 2003-0187052, 2003-0187257, 2003-0144300, 2003-0130254, 2003-0186974, 2003-0220352, 2003-0134876, 2004-0048903, 2004-0023945, 2004-0044036, 2004-0106641, 2004-0097593, 2004-0242643, 2004-0192701, 2004-0224971, 2004-0220183, 2004-0180900, 2004-0171798, 2004-0167199, 2004-0146561, 2004-0152754, 2004-0229918, 2005-0192336, 2005-0267196, 2005-0049258, 2006-0014782, 2006-0004003, 2006-0019932, 2005-0267196, 2005-0222207, 2005-0222207, 2006-0009481; PCT Publication No. WO 92/079778; and Molnar-Kimber, K. L. et al. J. Immunol., 150:295A (1993), each of which is incorporated herein by reference.

Type V Phosphodiesterase Inhibitors

Type V PDE inhibitors that can be used in the methods, compositions, and kits of the invention include those described in U.S. Pat. Nos. 6,992,192, 6,984,641, 6,960,587, 6,943,166, 6,878,711, and 6,869,950, and U.S. Patent Application Publication Nos. 2003-0144296, 2003-0171384, 2004-0029891, 2004-0038996, 2004-0186046, 2004-0259792, 2004-0087561, 2005-0054660, 2005-0042177, 2005-0245544, 2006-0009481, each of which is incorporated herein by reference.

Type VI Phosphodiesterase Inhibitors

Type VI PDE inhibitors that can be used in the methods, compositions, and kits of the invention include those described in U.S. Patent Application Publication Nos. 2004-0259792, 2004-0248957, 2004-0242673, and 2004-0259880, each of which is incorporated herein by reference.

Type VII Phosphodiesterase Inhibitors

Type VII PDE inhibitors that can be used in the methods, compositions, and kits of the invention include those described in the following patents, patent application, and references: U.S. Pat. Nos. 6,838,559, 6,753,340, 6,617,357, and 6,852,720; U.S. Patent Application Publication Nos. 2003-0186988, 2003-0162802, 2003-0191167, 2004-0214843, and 2006-0009481; PCT Publication WO 00/68230; and Martinez et al., J. Med. Chem. 43:683-689 (2000), each of which is incorporated herein by reference.

Non-Selective Phosphodiesterase Inhibitors

Non-selective PDE inhibitors that can be used in the methods, compositions, and kits of the invention include theophylline, papaverine, and ibudilast. Other PDE inhibitors that can be used in the methods, compositions, and kits of the invention are described in U.S. Pat. No. 6,953,774.

Corticosteroids

If desired, one or more corticosteroid may be administered in a method of the invention or may be formulated with a tetra-substituted pyrimidopyrimidine in a composition of the invention. Suitable corticosteroids include 11-alpha, 17-alpha,21 -trihydroxypregn-4-ene-3,20-dione; 11-beta,16-alpha,17,21 -tetrahydroxypregn-4-ene-3,20-dione; 11-beta,16-alpha,17,21 -tetrahydroxypregn-1,4-diene-3,20-dione; 11-beta,17-alpha,21-trihydroxy-6-alpha-methylpregn-4-ene-3,20-dione; 11-dehydrocorticosterone; 11-deoxycortisol; 11-hydroxy-1,4-androstadiene-3,17-dione; 11-ketotestosterone; 14-hydroxyandrost-4-ene-3,6,17-trione; 15,17-dihydroxyprogesterone; 16-methylhydrocortisone; 17,21 -dihydroxy-16-alpha-methylpregna-1,4,9(11)-triene-3,20-dione; 17-alpha-hydroxypregn-4-ene-3,20-dione; 17-alpha-hydroxypregnenolone; 17-hydroxy-16-beta-methyl-5-beta-pregn-9(11)-ene-3,20-dione; 17-hydroxy-4,6,8(14)-pregnatriene-3,20-dione; 17-hydroxypregna-4,9(11)-diene-3,20-dione; 18-hydroxycorticosterone; 18-hydroxycortisone; 18-oxocortisol; 21-acetoxypregnenolone; 21-deoxyaldosterone; 21-deoxycortisone; 2-deoxyecdysone; 2-methylcortisone; 3-dehydroecdysone; 4-pregnene-17-alpha,20-beta, 21-triol-3,11-dione; 6,17,20-trihydroxypregn-4-ene-3-one; 6-alpha-hydroxycortisol; 6-alpha-fluoroprednisolone, 6-alpha-methylprednisolone, 6-alpha-methylprednisolone 21-acetate, 6-alpha-methylprednisolone 21-hemisuccinate sodium salt, 6-beta-hydroxycortisol, 6-alpha, 9-alpha-difluoroprednisolone 21-acetate 17-butyrate, 6-hydroxycorticosterone; 6-hydroxydexamethasone; 6-hydroxyprednisolone; 9-fluorocortisone; alclomethasone dipropionate; aldosterone; algestone; alphaderm; amadinone; amcinonide; anagestone; androstenedione; anecortave acetate; beclomethasone; beclomethasone dipropionate; betamethasone 17-valerate; betamethasone sodium acetate; betamethasone sodium phosphate; betamethasone valerate; bolasterone; budesonide; calusterone; chlormadinone; chloroprednisone; chloroprednisone acetate; cholesterol; ciclesonide; clobetasol; clobetasol propionate; clobetasone; clocortolone; clocortolone pivalate; clogestone; cloprednol; corticosterone; cortisol; cortisol acetate; cortisol butyrate; cortisol cypionate; cortisol octanoate; cortisol sodium phosphate; cortisol sodium succinate; cortisol valerate; cortisone; cortisone acetate; cortivazol; cortodoxone; daturaolone; deflazacort, 21-deoxycortisol, dehydroepiandrosterone; delmadinone; deoxycorticosterone; deprodone; descinolone; desonide; desoximethasone; dexafen; dexamethasone; dexamethasone 21-acetate; dexamethasone acetate; dexamethasone sodium phosphate; dichlorisone; diflorasone; diflorasone diacetate; diflucortolone; difluprednate; dihydroelatericin a; domoprednate; doxibetasol; ecdysone; ecdysterone; emoxolone; endrysone; enoxolone; fluazacort; flucinolone; flucloronide; fludrocortisone; fludrocortisone acetate; flugestone; flumethasone; flumethasone pivalate; flumoxonide; flunisolide; fluocinolone; fluocinolone acetonide; fluocinonide; fluocortin butyl; 9-fluorocortisone; fluocortolone; fluorohydroxyandrostenedione; fluorometholone; fluorometholone acetate; fluoxymesterone; fluperolone acetate; fluprednidene; fluprednisolone; flurandrenolide; fluticasone; fluticasone propionate; formebolone; formestane; formocortal; gestonorone; glyderinine; halcinonide; halobetasol propionate; halometasone; halopredone; haloprogesterone; hydrocortamate; hydrocortiosone cypionate; hydrocortisone; hydrocortisone 21-butyrate; hydrocortisone aceponate; hydrocortisone acetate; hydrocortisone buteprate; hydrocortisone butyrate; hydrocortisone cypionate; hydrocortisone hemisuccinate; hydrocortisone probutate; hydrocortisone sodium phosphate; hydrocortisone sodium succinate; hydrocortisone valerate; hydroxyprogesterone; inokosterone; isoflupredone; isoflupredone acetate; isoprednidene; loteprednol etabonate; meclorisone; mecortolon; medrogestone; medroxyprogesterone; medrysone; megestrol; megestrol acetate; melengestrol; meprednisone; methandrostenolone; methylprednisolone; methylprednisolone aceponate; methylprednisolone acetate; methylprednisolone hemisuccinate; methylprednisolone sodium succinate; methyltestosterone; metribolone; mometasone; mometasone furoate; mometasone furoate monohydrate; nisone; nomegestrol; norgestomet; norvinisterone; oxymesterone; paramethasone; paramethasone acetate; ponasterone; prednicarbate; prednisolamate; prednisolone; prednisolone 21-diethylaminoacetate; prednisolone 21-hemisuccinate; prednisolone acetate; prednisolone famesylate; prednisolone hemisuccinate; prednisolone-21(beta-D-glucuronide); prednisolone metasulphobenzoate; prednisolone sodium phosphate; prednisolone steaglate; prednisolone tebutate; prednisolone tetrahydrophthalate; prednisone; prednival; prednylidene; pregnenolone; procinonide; tralonide; progesterone; promegestone; rhapontisterone; rimexolone; roxibolone; rubrosterone; stizophyllin; tixocortol; topterone; triamcinolone; triamcinolone acetonide; triamcinolone acetonide 21-palmitate; triamcinolone benetonide; triamcinolone diacetate; triamcinolone hexacetonide; trimegestone; turkesterone; and wortmannin.

Standard recommended dosages for corticosteroids are provided, e.g., in the Merck Manual of Diagnosis & Therapy (17th Ed. M H Beers et al., Merck & Co.) and Physicians' Desk Reference 2003 (57^{th} Ed. Medical Economics Staff et al., Medical Economics Co., 2002). In one embodiment, the dosage of corticosteroid administered is a dosage equivalent to a prednisolone dosage, as defined herein. For example, a low dosage of a corticosteroid may be considered as the dosage equivalent to a low dosage of prednisolone.

Steroid Receptor Modulators

Steroid receptor modulators (e.g., antagonists and agonists) may be used as a substitute for or in addition to a corticosteroid in the methods, compositions, and kits of the invention. Thus, in one embodiment, the invention features the combination of a tetra-substituted pyrimidopyrimidine or an adenosine activity upregulator and a glucocorticoid receptor modulator or other steroid receptor modulator, and methods of treating immunoinflammatory disorders therewith.

Glucocorticoid receptor modulators that may used in the methods, compositions, and kits of the invention include compounds described in U.S. Pat. Nos. 6,380,207, 6,380,223, 6,448,405, 6,506,766, and 6,570,020, U.S. Patent Application Publication Nos. 2003-0176478, 2003-0171585, 2003-0120081, 2003-0073703, 2002-015631, 2002-0147336, 2002-0107235, 2002-0103217, and 2001-0041802, and PCT Publication No. WO 00/66522, each of which is hereby incorporated by reference. Other steroid receptor modulators may also be used in the methods, compositions, and kits of the invention are described in U.S. Pat. Nos. 6,093,821, 6,121,450, 5,994,544, 5,696,133, 5,696,127, 5,693,647, 5,693,646, 5,688,810, 5,688,808, and 5,696,130, each of which is hereby incorporated by reference.

Other Compounds

Other compounds that may be used as a substitute for or in addition to a corticosteroid in the methods, compositions, and kits of the invention A-348441 (Karo Bio), adrenal cortex extract (GlaxoSmithKline), alsactide (Aventis), amebucort (Schering AG), amelometasone (Taisho), ATSA (Pfizer), bitolterol (Elan), CBP-2011 (InKine Pharmaceutical), cebaracetam (Novartis) CGP-13774 (Kissei), ciclesonide (Altana), ciclometasone (Aventis), clobetasone butyrate (GlaxoSmithKline), cloprednol (Hoffmann-La Roche), collismycin A (Kirin), cucurbitacin E (NIH), deflazacort (Aventis), deprodone propionate (SSP), dexamethasone acefurate (Schering-Plough), dexamethasone linoleate (GlaxoSmithKline), dexamethasone valerate (Abbott), difluprednate (Pfizer), domoprednate (Hoffinann-La Roche), ebiratide (Aventis), etiprednol dicloacetate (IVAX), fluazacort (Vicuron), flumoxonide (Hoffinann-La Roche), fluocortin butyl (Schering AG), fluocortolone monohydrate (Schering AG), GR-250495X (GlaxoSmithKline), halometasone (Novartis), halopredone (Dainippon), HYC-141 (Fidia), icomethasone enbutate (Hovione), itrocinonide (AstraZeneca), L-6485 (Vicuron), Lipocort (Draxis Health), locicortone (Aventis), meclorisone (Schering-Plough), naflocort (Bristol-Myers Squibb), NCX-1015 (NicOx), NCX-1020 (NicOx), NCX-1022 (NicOx), nicocortonide (Yamanouchi), NIK-236 (Nikken Chemicals), NS-126 (SSP), Org-2766 (Akzo Nobel), Org-6632 (Akzo Nobel), P16CM, propylmesterolone (Schering AG), RGH-1113 (Gedeon Richter), rofleponide (AstraZeneca), rofleponide palmitate (AstraZeneca), RPR-106541 (Aventis), RU-26559 (Aventis), Sch-19457 (Schering-Plough), T25 (Matrix Therapeutics), TBI-PAB (Sigma-Tau), ticabesone propionate (Hoffmann-La Roche), tifluadom (Solvay), timobesone (Hoffmann-La Roche), TSC-5 (Takeda), and ZK-73634 (Schering AG).

Non-Steroidal Anti-Inflammatory Drugs (NSAIDs)

If desired, the tetra-substituted pyrimidopyrimidine or adenosine activity upregulator of the invention may be administered in conjunction with one or more of non-steroidal anti-inflammatory drugs (NSAIDs), such as naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid (salsalate), fenoprofen, flurbiprofen, ketoprofen, meclofenamate sodium, meloxicam, oxaprozin, sulindac, and tolmetin.

An NSAID may be administered in conjunction with any one of the combinations described in this application. For example, a patient suffering from periodontal disease or having an increased serum CRP level may be initially treated with a combination of a tetra-substituted pyrimidopyrimidine and a corticosteroid and then treated with an NSAID, such as acetylsalicylic acid, in conjunction with the combination described above. Dosage amounts of acetylsalicylic acid are known to those skilled in medical arts, and generally range from about 70 mg to about 350 mg per day.

Nonsteroidal Immunophilin-Dependent Immunosuppressants

In one embodiment, the invention features methods, compositions, and kits employing a tetra-substituted pyrimidopyrimidine or an adenosine activity upregulator and a non-steroidal immunophilin-dependent immunosuppressant (NsIDI), optionally with a corticosteroid or other agent described herein.

In one embodiment, the NsIDI is cyclosporine, and is administered in an amount between 0.05 and 50 milligrams per kilogram per day (e.g., orally in an amount between 0.1 and 12 milligrams per kilogram per day). In another embodiment, the NsIDI is tacrolimus and is administered in an amount between 0.0001-20 milligrams per kilogram per day (e.g., orally in an amount between 0.01-0.2 milligrams per kilogram per day). In another embodiment, the NsIDI is rapamycin and is administered in an amount between 0.1-502 milligrams per day (e.g., at a single loading dose of 6 mg/day, followed by a 2 mg/day maintenance dose). In another embodiment, the NsIDI is everolimus, administered at a dosage of 0.75-8 mg/day. In still other embodiments, the NsIDI is pimecrolimus, administered in an amount between 0.1 and 200 milligrams per day (e.g., as a 1% cream/twice a day to treat atopic dermatitis or 60 mg a day for the treatment of psoriasis), or the NsIDI is a calcineurin-binding peptide administered in an amount and frequency sufficient to treat the patient. Two or more NsIDIs can be administered contemporaneously.

Cyclosporines

The cyclosporines are fungal metabolites that comprise a class of cyclic oligopeptides that act as immunosuppressants. Cyclosporine A is a hydrophobic cyclic polypeptide consisting of eleven amino acids. It binds and forms a complex with the intracellular receptor cyclophilin. Thecyclosporine/cyclophilin complex binds to and inhibits calcineurin, a Ca²⁺-calmodulin-dependent serine-threonine-specific protein phosphatase. Calcineurin mediates signal transduction events required for T-cell activation (reviewed in Schreiber et al., Cell 70:365-368, 1991). Cyclosporines and their functional and structural analogs suppress the T cell-dependent immune response by inhibiting antigen-triggered signal transduction. This inhibition decreases the expression of proinflammatory cytokines, such as IL-2.

Many different cyclosporines (e.g., cyclosporine A, B, C, D, E, F, G, H, and I) are produced by fungi. Cyclosporine A is a commercially available under the trade name NEORAL from Novartis. Cyclosporine A structural and functional analogs include cyclosporines having one or more fluorinated amino acids (described, e.g., in U.S. Pat. No. 5,227,467); cyclosporines having modified amino acids (described, e.g., in U.S. Pat. Nos. 5,122,511 and 4,798,823); and deuterated cyclosporines, such as ISAtx247 (described in U.S. Patent Application Publication No. 2002/0132763 A1). Additional cyclosporine analogs are described in U.S. Pat. Nos. 6,136,357, 4,384,996, 5,284,826, and 5,709,797. Cyclosporine analogs include, but are not limited to, D-Sar (α-SMe)³ Val²-DH-Cs (209-825), Allo-Thr-2-Cs, Norvaline-2-Cs, D-Ala(3-acetylamino)-8-Cs, Thr-2-Cs, and D-MeSer-3-Cs, D-Ser(O—CH₂CH₂—OH)-8-Cs, and D-Ser-8-Cs, which are described in Cruz et al. (Antimicrob. Agents Chemother. 44:143-149, 2000).

Cyclosporines are highly hydrophobic and readily precipitate in the presence of water (e.g. on contact with body fluids). Methods of providing cyclosporine formulations with improved bioavailability are described in U.S. Pat. Nos. 4,388,307, 6,468,968, 5,051,402, 5,342,625, 5,977,066, and 6,022,852. Cyclosporine microemulsion compositions are described in U.S. Pat. Nos. 5,866,159, 5,916,589, 5,962,014, 5,962,017, 6,007,840, and 6,024,978.

Cyclosporines can be administered intravenously, topically, or orally, but oral administration is preferred. To overcome the hydrophobicity of cyclosporine A, an intravenous cyclosporine A may be provided in an ethanol-polyoxyethylated castor oil vehicle that must be diluted prior to administration. Cyclosporine A may be provided, e.g., as a microemulsion in a 25 mg or 100 mg tablets, or in a 100 mg/ml oral solution (NEORAL).

Tacrolimus

Tacrolimus (FK506) is an immunosuppressive agent that targets T cell intracellular signal transduction pathways. Tacrolimus binds to an intracellular protein FK506 binding protein (FKBP-12) that is not structurally related to cyclophilin (Harding et al. Nature 341:758-7601, 1989; Siekienka et al. Nature 341:755-757, 1989; and Soltoffet al., J. Biol. Chem. 267:17472-17477, 1992). The FKBP/FK506 complex binds to calcineurin and inhibits calcineurin's phosphatase activity. This inhibition prevents the dephosphorylation and nuclear translocation of nuclear factor of activated T cells (NFAT), a nuclear component that initiates gene transcription required for proinflammatory cytokine (e.g., IL-2, gamma interferon) production and T cell activation. Thus, tacrolimus inhibits T cell activation.

Tacrolimus is a macrolide antibiotic that is produced by Streptomyces tsukubaensis. It suppresses the immune system and prolongs the survival of transplanted organs. It is currently available in oral, topical, and injectable formulations. Tacrolimus capsules contain 0.5 mg, 1 mg, or 5 mg of anhydrous tacrolimus within a gelatin capsule shell. The injectable formulation contains 5 mg anhydrous tacrolimus in castor oil and alcohol that is diluted with 0.9% sodium chloride or 5% dextrose prior to injection. While oral administration is preferred, patients unable to take oral capsules may receive injectable tacrolimus. The initial dose should be administered no sooner than six hours after transplant by continuous intravenous infusion.

Tacrolimus and tacrolimus analogs are described by Tanaka et al., (J. Am. Chem. Soc., 109:5031, 1987) and in U.S. Pat. Nos. 4,894,366, 4,929,611, and 4,956,352. FK506-related compounds, including FR-900520, FR-900523, and FR-900525, are described in U.S. Pat. No. 5,254,562; O-aryl, O-alkyl, O-alkenyl, and O-alkynylmacrolides are described in U.S. Pat. Nos. 5,250,678, 532,248, 5,693,648; amino O-aryl macrolides are described in U.S. Pat. No. 5,262,533; alkylidene macrolides are described in U.S. Pat. No. 5,284,840; N-heteroaryl, N-alkylheteroaryl, N-alkenylheteroaryl, and N-alkynylheteroaryl macrolides are described in U.S. Pat. No. 5,208,241; aminomacrolides and derivatives thereof are described in U.S. Pat. No. 5,208,228; fluoromacrolides are described in U.S. Pat. No. 5,189,042; amino O-alkyl, O-alkenyl, and O-alkynylmacrolides are described in U.S. Pat. No. 5,162,334; and halomacrolides are described in U.S. Pat. No. 5,143,918.

Tacrolimus is extensively metabolized by the mixed-function oxidase system, in particular, by the cytochrome P-450 system. The primary mechanism of metabolism is demethylation and hydroxylation. While various tacrolimus metabolites are likely to exhibit immunosuppressive biological activity, the 13-demethyl metabolite is reported to have the same activity as tacrolimus.

Pimecrolimus

Pimecrolimus is the 33-epi-chloro derivative of the macrolactam ascomyin. Pimecrolimus structural and functional analogs are described in U.S. Pat. No. 6,384,073. Pimecrolimus is particularly useful for the treatment of atopic dermatitis. Pimecrolimus is currently available as a 1% cream. Oral pimecrolimus can be given in amounts of 40-240 mg/day.

Rapamycin

Rapamycin is a cyclic lactone produced by Streptomyces hygroscopicus. Rapamycin is an immunosuppressive agent that inhibits T cell activation and proliferation. Like cyclosporines and tacrolimus, rapamycin forms a complex with the immunophilin FKBP-12, but the rapamycin-FKBP-12 complex does not inhibit calcineurin phosphatase activity. The rapamycin immunophilin complex binds to and inhibits the mammalian kinase target of rapamycin (mTOR). mTOR is a kinase that is required for cell-cycle progression. Inhibition of mTOR kinase activity blocks T cell activation and proinflammatory cytokine secretion.

Rapamycin structural and functional analogs include mono- and diacylated rapamycin derivatives (U.S. Pat. No. 4,316,885); rapamycin water-soluble prodrugs (U.S. Pat. No. 4,650,803); carboxylic acid esters (PCT Publication No. WO 92/05179); carbamates (U.S. Pat. No. 5,118,678); amide esters (U.S. Pat. No. 5,118,678); biotin esters (U.S. Pat. No. 5,504,091); fluorinated esters (U.S. Pat. No. 5,100,883); acetals (U.S. Pat. No. 5,151,413); silyl ethers (U.S. Pat. No. 5,120,842); bicyclic derivatives (U.S. Pat. No. 5,120,725); rapamycin dimers (U.S. Pat. No. 5,120,727); O-aryl, O-alkyl, O-alkyenyl and O-alkynyl derivatives (U.S. Pat. No. 5,258,389); and deuterated rapamycin (U.S. Pat. No. 6,503,921). Additional rapamycin analogs are described in U.S. Pat. Nos. 5,202,332 and 5,169,851.

Rapamycin is currently available for oral administration in liquid and tablet formulations. RAPAMUNE liquid contains 1 mg/mL rapamycin that is diluted in water or orange juice prior to administration. Tablets containing 1 or 2 mg of rapamycin are also available. Rapamycin is preferably given once daily. It is absorbed rapidly and completely after oral administration. Typically, patient dosage of rapamycin varies according to the patient's condition, but some standard recommended dosages are provided below. The initial loading dose for rapamycin is 6 mg. Subsequent maintenance doses of 0.5-2 mg/day are typical. Alternatively, a loading dose of 3 mg, 5 mg, 10 mg, 15 mg, 20 mg, or 25 mg can be used with a 1 mg, 3 mg, 5 mg, 7 mg, or 10 mg per day maintenance dose. In patients weighing less than 40 kg, rapamycin dosages are typically adjusted based on body surface area; generally a 3 mg/m²/day loading dose and a 1 mg/m²/day maintenance dose is used.

Peptide Moieties

Peptides, peptide mimetics, peptide fragments, either natural, synthetic or chemically modified, that impair the calcineurin-mediated dephosphorylation and nuclear translocation of NFAT are suitable for use in practicing the invention. Examples of peptides that act as calcineurin inhibitors by inhibiting the NFAT activation and the NFAT transcription factor are described, e.g., by Aramburu et al., Science 285:2129-2133, 1999) and Aramburu et al., Mol. Cell 1:627-637, 1998). As a class of calcineurin inhibitors, these agents are useful in the methods of the invention.

Cotherapy

If desired, one or more additional agents may be administered in conjunction with the methods of the invention. Suitable agents include antibiotics (minocycline, penicillin, cephalosporin, tetracycline, oxytetracycline, chlortetracycline, metronidazole, chloramphenicol, streptomycin, neomycin, sulfonamides, phenolic compounds, quartemary ammonium compounds, doxycycline); antiseptics (e.g., chlorhexidine); nonsteroidal antiinflammatories (e.g., flurbiprofen, carprofen, diclofenac, fenbufen, fenclozic acid, fenoprofen, flufenamic acid, ibuprofen, indomethacin, indoprofen, ketoprofen, lonazolac, loxoprofen, meclofenamic acid, mefanamic acid, naproxen, proprionic acids, salicylic acids, sulindac, tolmetin, meloxicam, oxicams, piroxicam, tenoxicam, etodolac, and oxaprozin); tranexamic acid, allantoin; epsilon-aminocaproic acid; lysozyme; dihydrocholesterol; beta-glycyrrhetinic acid; platelet aggregation inhibitors (e.g., abciximab, aspirin, cilostazol, clopidogrel, eptifibatide, ticlopidine, or tirofiban); anticoagulants (e.g., dalteparin, danaparoid, enoxaparin, heparin, tinzaparin, or warfarin); antipyretics (e.g., acetaminophen); ticlopidine; clopidogrel; angiotensin converting enzyme inhibitors; beta blockers; pentoxifylline; cilostazol; estrogen replacement therapy; and lipid-lowering agents (e.g., cholestyramine, colestipol, nicotinic acid, gemfibrozil, probucol, ezetimibe, or statins such as atorvastatin, rosuvastatin, lovastatin simvastatin, pravastatin, cerivastatin, and fluvastatin). These agents may be administered concomitantly or within 14 days of the method of the invention. If desired, one or more of the foregoing agents is coformulated with one or more agents of the invention to form a single composition. Thus, in one embodiment, the invention features a tetra-substituted pyrimidopyrimidine, one of the foregoing agents, and, optionally, a corticosteroid.

Dosages

The dosage of each compound of the claimed combinations depends on several factors, including: the administration method, the disease to be treated, the severity of the disease, whether the disease is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect dosage used.

Continuous daily dosing with the combinations of the invention may not be required. A therapeutic regimen may require cycles, during which time a drug is not administered, or therapy may be provided on an as needed basis during periods of acute inflammation.

As described above, the compound in question may be administered orally in the form of tablets, capsules, elixirs or syrups, or rectally in the form of suppositories. Parenteral administration of a compound is suitably performed, for example, in the form of saline solutions or with the compound incorporated into liposomes. In cases where the compound in itself is not sufficiently soluble to be dissolved, a solubilizer such as ethanol can be applied.

Devices

One or more agents of the invention (e.g., amoxapine and/or dipyridamole) can be delivered to the periodontal pockets of a patient by way of a drug delivery device. Such devices are known in the art (see, e.g., U.S. Pat. Nos. 4,685,883; 5,262,164; 5,366,733; 5,447,725; 5,599,553; and 5,939,047).

The following examples are to illustrate the invention. They are not meant to limit the invention in any way.

### EXAMPLES

Study Protocol

We conducted a seven week blinded, randomized study with daily oral administration of the combination of prednisolone and dipyridamole or placebo, with regular CRP and inflammatory cytokine measurements. The study population had severe periodontitis with at least 10 pockets of ≧5 mm in depth, with at least four pockets ≧6-9 mm. To qualify, 10% of the pockets must bleed on probing. The subject must otherwise have been in good general health.

During the study, the subjects were seen at the following study visits:
Screening visit (visit 1)
Day 1 (Baseline visit/visit 2)
Day 7±0 day (visit 3)
Day 14±0 day (visit 4)
Day 28±1 day (visit 5)
Day 42±1 day (visit 6)
Day 49±1 day (end of study visit/visit 7)
   Subjects were evaluated for study eligibility at the Screening visit, which was conducted within 14 days before the first dose of study drug. Treated subjects received scaling and root planing (SRP) treatment after 42 days on study drug, after pocket depth determination. All study subjects continued on study medication for an additional one week, after which serum CRP, IL-6, and IFN-γ levels were determined on day 49.

Subjects were randomized into treatment groups and received either dipyridamole and prednisolone at the doses indicated below or placebo tablets. In the treatment group there was one dose escalations after Day 14 as follows:
Days 1-14: Dose Level 1 (200 mg dipyridamole and 3 mg prednisolone)
Days 15-49: Dose Level 2 (400 mg dipyridamole and 3 mg prednisolone)

The drugs were blister packed as follows:

| | | |
|---|---|---|
| 8 am | 8 am | 1 pm |

Treatment group

| | | | |
|---|---|---|---|
| Day 1-14 | 1 mg prednisolone | 100 mg | 100 mg dipyridamole |
| | 1 mg prednisolone | dipyridamole | 1 mg prednisolone |
| Day 15-49 | 1 mg prednisolone | 100 mg | 100 mg dipyridamole |
| | | dipyridamole | |
| | 1 mg prednisolone | 100 mg | 100 mg dipyridamole |
| | | dipyridamole | 1 mg prednisolone |

Placebo

| | | | |
|---|---|---|---|
| Day 1-49 | Placebo (white) | Placebo (blue) | Placebo (white) |
| | Placebo (white) | Placebo (blue) | Placebo (blue) |
| | | | Placebo (blue) |

The serum CRP level, periodontal pocket depth, IFN-γ level, and IL-6 level were determined using standard techniques. The results are shown in Tables 1-3. In these tables, (a) indicated the p-value derived from a Wilcoxon Rank sum test, which tests if the center of the distribution of change scores is significantly lower in the treatment group than the placebo group. Study Baseline represents pre-treatment, while SRP Baseline represents end of SRP therapy at Day 42.

Fifty-seven patients were enrolled at one study center in Sweden and randomized to one of two treatment arms. Sixty-one percent were males and 39% were females. The overall age was 57 years.

The primary endpoint of change in CRP from Baseline to Day 42 was significantly lower for the patients receiving treatment compared to the patients receiving placebo (p=0.0241). These results were derived from a Wilcoxon Rank-Sum test to test if the center of the distribution of change scores in CRP was significantly lower in the treatment group compared to the placebo group. This primary analysis excluded any CRP value that was collected on the same date as the occurrence of an upper respiratory tract infection captured as an adverse event. The median change from Baseline to Day 42 in CRP was −0.65 for the patients receiving treatment and −0.10 for the patients receiving placebo.

The primary endpoint of change in IL-6 from Baseline to Day 42 was significantly lower for the patients receiving treatment compared to the patients receiving placebo (p=0.0375). These results were derived from a Wilcoxon Rank-Sum test to test if the center of the distribution of change scores in IL-6 was significantly lower in the treatment group compared to the placebo group. The median change from Baseline to Day 42 in IL-6 was 0.00 for the patients receiving treatment and 1.00 for the patients receiving placebo.

The primary endpoint of change in IFN-γ from Baseline to Day 42 was significantly lower for the patients receiving treatment compared to the patients receiving placebo (p=0.0241). These results were derived from a Wilcoxon Rank-Sum test to test if the center of the distribution of change scores in IFN-γ was significantly lower in the treatment group compared to the placebo group. The median change from Baseline to Day 42 in IFN-γ was −1.00 for the patients receiving treatment and 0.59 for the patients receiving placebo.

TABLE 1
CRP

| | Placebo N = 29 | | | Treatment N = 28 | | |
|---|---|---|---|---|---|---|
| | | Change from | Change from | | Change from | Change from |
| Visit | Actual Value | Study Baseline | SRP Baseline | Actual Value | Study Baseline | SRP Baseline |
| Study Baseline | | | | | | |
| N | 29 | | | 28 | | |
| Mean (Std Dev) | 4.19 (2.799) | | | 6.42 (13.698) | | |
| Median | 3.80 | | | 3.20 | | |
| Min, Max | 0.8, 12.0 | | | 0.7, 74.8 | | |
| Day 7 | | | | | | |
| N | 27 | 27 | | 28 | 28 | |
| Mean (Std Dev) | 4.45 (3.081) | 0.28 (1.755) | | 3.72 (4.504) | −2.70 (13.843) | |
| Median | 3.50 | 0.40 | | 2.55 | −0.75 | |
| Min, Max | 1.0, 14.0 | −3.0, 4.0 | | 0.6, 24.6 | −68.7, 19.8 | |
| Day 14 | | | | | | |
| N | 29 | 29 | | 28 | 28 | |
| Mean (Std Dev) | 4.07 (2.955) | −0.12 (3.000) | | 2.84 (1.387) | −3.59 (13.918) | |
| Median | 3.40 | −0.10 | | 2.60 | −0.70 | |
| Min, Max | 0.8, 15.8 | −8.5, 10.0 | | 0.5, 6.5 | −73.7, 2.4 | |
| Day 28 | | | | | | |
| N | 29 | 29 | | 28 | 28 | |
| Mean (Std Dev) | 5.14 (4.700) | 0.95 (4.176) | | 3.92 (3.452) | −2.50 (14.211) | |
| Median | 3.50 | 0.20 | | 3.30 | −0.35 | |
| Min, Max | 0.9, 18.8 | −8.2, 14.9 | | 0.5, 17.0 | −72.9, 13.7 | |
| Day 42 (SRP Baseline) | | | | | | |
| N | 29 | 29 | | 28 | 28 | |
| Mean (Std Dev) | 5.68 (9.330) | 1.50 (9.639) | | 2.82 (1.599) | −3.60 (12.919) | |
| Median | 3.60 | −0.10 | | 2.50 | −0.65 | |
| Min, Max | 0.8, 52.7 | −8.0, 50.5 | | 0.7, 6.7 | −68.1, 2.2 | |
| One Sided p-value (a) | | | | | 0.0241 | |
| Day 49 | | | | | | |
| N | 29 | 29 | 29 | 28 | 28 | 28 |
| Mean (Std Dev) | 5.47 (8.263) | 1.29 (7.438) | −0.21 (11.717) | 4.22 (5.987) | −2.20 (15.512) | 1.40 (6.117) |
| Median | 3.30 | −0.10 | 0.10 | 2.75 | −0.25 | 0.20 |
| Min, Max | 1.0, 46.5 | −8.8, 36.8 | −49.8, 35.4 | 0.6, 32.8 | −73.4, 31.8 | −5.3, 31.4 |
| One Sided p-value (a) | | | | | 0.1983 | 0.2706 |

TABLE 2
IL-6

| | Placebo N = 29 | | | Treatment N = 28 | | |
|---|---|---|---|---|---|---|
| | | Change from | Change from | | Change from | Change from |
| Visit | Actual Value | Study Baseline | SRP Baseline | Actual Value | Study Baseline | SRP Baseline |
| Study Baseline | | | | | | |
| N | 29 | | | 28 | | |
| Mean (Std Dev) | 5.17 (1.823) | | | 10.86 (26.132) | | |
| Median | 5.00 | | | 5.00 | | |
| Min, Max | 2.8, 10.0 | | | 3.0, 143.0 | | |
| Day 7 | | | | | | |
| N | 29 | 29 | | 28 | 28 | |
| Mean (Std Dev) | 5.45 (1.744) | 0.28 (1.578) | | 9.96 (19.709) | −0.90 (12.041) | |
| Median | 5.00 | 0.00 | | 5.00 | −0.10 | |
| Min, Max | 3.0, 10.0 | −5.0, 3.0 | | 2.8, 95.0 | −48.0, 39.0 | |
| One Sided p-value (a) | | | | | 0.0159 | |
| Day 14 | | | | | | |
| N | 29 | 29 | | 28 | 28 | |
| Mean (Std Dev) | 5.76 (2.370) | 0.59 (2.470) | | 9.93 (22.198) | −0.93 (4.472) | |
| Median | 5.00 | 0.00 | | 5.00 | 0.00 | |
| Min, Max | 3.0, 13.0 | −5.0, 8.0 | | 3.0, 122.0 | −21.0, 4.0 | |
| One Sided p-value (a) | | | | | 0.1008 | |
| Day 28 | | | | | | |
| N | 29 | 29 | | 28 | 28 | |
| Mean (Std Dev) | 5.79 (2.455) | 0.63 (2.822) | | 10.10 (21.178) | −0.76 (5.467) | |
| Median | 5.00 | 0.00 | | 5.50 | 0.00 | |
| Min, Max | 3.0, 14.0 | −5.0, 10.0 | | 2.8, 117.0 | −26.0, 7.0 | |
| One Sided p-value (a) | | | | | 0.1674 | |
| Day 42 (SRP Baseline) | | | | | | |
| N | 29 | 29 | | 28 | 28 | |
| Mean (Std Dev) | 5.40 (1.823) | 0.23 (2.106) | | 9.06 (18.699) | −1.79 (7.579) | |
| Median | 5.00 | 1.00 | | 4.50 | 0.00 | |
| Min, Max | 2.8, 10.0 | −5.0, 6.0 | | 2.8, 103.0 | −40.0, 1.0 | |
| One Sided p-value (a) | | | | | 0.0375 | |
| Day 49 | | | | | | |
| N | 29 | 29 | 29 | 28 | 28 | 28 |
| Mean (Std Dev) | 5.67 (2.504) | 0.50 (2.288) | 0.27 (2.297) | 8.83 (16.498) | −2.23 (10.006) | −0.44 (3.384) |
| Median | 5.00 | 1.00 | 0.00 | 5.00 | −0.10 | 0.00 |
| Min, Max | 2.8, 11.0 | −5.0, 6.0 | −5.0, 6.0 | 2.8, 92.0 | −51.0, 6.0 | −11.0, 5.0 |
| One Sided p-value (a) | | | | | 0.0709 | 0.4968 |

TABLE 3
IFN-γ

| | Placebo N = 29 | | | Treatment N = 28 | | |
|---|---|---|---|---|---|---|
| | | Change from | Change from | | Change from | Change from |
| Visit | Actual Value | Study Baseline | SRP Baseline | Actual Value | Study Baseline | SRP Baseline |
| Study Baseline | | | | | | |
| N | 29 | | | 28 | | |
| Mean (Std Dev) | 9.919 (1.2947) | | | 11.315 (3.3956) | | |
| Median | 9.870 | | | 10.500 | | |
| Min, Max | 7.81, 13.00 | | | 8.24, 26.00 | | |
| Day 7 | | | | | | |
| N | 29 | 29 | | 28 | 28 | |
| Mean (Std Dev) | 10.935 (3.2962) | 1.016 (3.1534) | | 10.749 (3.8789) | −0.566 (1.7944) | |
| Median | 9.690 | 0.180 | | 9.715 | 0.000 | |
| Min, Max | 8.06, 21.00 | −2.47, 12.19 | | 7.08, 28.00 | −5.92, 3.00 | |
| One Sided p-value (a) | | | | | 0.0464 | |
| Day 14 | | | | | | |
| N | 29 | 29 | | 28 | 28 | |
| Mean (Std Dev) | 10.369 (2.6117) | 0.450 (2.2715) | | 10.857 (3.8021) | −0.458 (2.8078) | |
| Median | 9.740 | −0.190 | | 9.600 | −0.605 | |
| Min, Max | 7.62, 20.00 | −2.47, 7.00 | | 7.56, 26.00 | −4.82, 8.00 | |
| One Sided p-value (a) | | | | | 0.0511 | |
| Day 28 | | | | | | |
| N | 29 | 29 | | 28 | 28 | |
| Mean (Std Dev) | 10.151 (2.2305) | 0.232 (2.6546) | | 10.843 (3.9230) | −0.472 (2.4011) | |
| Median | 9.280 | −0.700 | | 9.510 | −0.355 | |
| Min, Max | 7.52, 16.00 | −3.00, 7.76 | | 7.44, 27.00 | −7.56, 4.76 | |
| One Sided p-value (a) | | | | | 0.3515 | |
| Day 42 (SRP Baseline) | | | | | | |
| N | 29 | 29 | | 28 | 28 | |
| Mean (Std Dev) | 10.799 (2.2471) | 0.880 (2.5147) | | 10.771 (3.6819) | −0.544 (2.0194) | |
| Median | 11.000 | 0.590 | | 9.225 | −1.000 | |
| Min, Max | 7.70, 16.00 | −3.24, 5.57 | | 7.35, 25.00 | −4.10, 4.76 | |
| One Sided p-value (a) | | | | | 0.0277 | |
| Day 49 | | | | | | |
| N | 29 | 29 | 29 | 28 | 28 | 28 |
| Mean (Std Dev) | 11.355 (2.7354) | 1.436 (3.2354) | 0.556 (1.8455) | 11.094 (3.7908) | −0.221 (2.5603) | 0.323 (1.8107) |
| Median | 11.000 | 1.000 | 0.760 | 9.795 | 0.000 | 0.290 |
| Min, Max | 6.99, 17.00 | −4.10, 7.57 | −4.00, 3.94 | 7.44, 26.00 | −5.00, 5.00 | −4.00, 4.76 |
| One Sided p-value (a) | | | | | 0.0216 | 0.2373 |

### Other Embodiments

Various modifications and variations of the described methods and compositions of the invention will be apparent to those skilled in the art without departing from the scope and spirit of the invention. Although the invention has been described in connection with specific desired embodiments, it should be understood that the invention as claimed should not be unduly limited to such specific embodiments. Indeed, various modifications of the described modes for carrying out the invention that are obvious to those skilled in the fields of medicine, immunology, pharmacology, endocrinology, or related fields are intended to be within the scope of the invention.

All publications mentioned in this specification are herein incorporated by reference to the same extent as if each independent publication was specifically and individually incorporated by reference.

What is claimed is:

1. A method for treating a disease or condition associated with an increased serum CRP, IL-6, and/or IFN-γ levels in a patient in need thereof, said method comprising administering to said patient (i) a corticosteroid; and (ii) a tetra-substituted pyrimidopyrimidine, wherein said corticosteroid and tetra-substituted pyrimidopyrimidine are administered in amounts and for a duration that together are sufficient to reduce serum CRP, IL-6, and/or IFN-γ levels in said patient.

2. The method of claim 1, wherein said tetra-substituted pyrimidopyrimidine is selected from the group consisting of 2,6-disubstituted 4,8-dibenzylaminopyrimido[5,4-d]pyrimidines; mopidamole; dipyridamole monoacetate; 1-((2,7-bis(2-methyl-4-morpholinyl)-6-phenyl-4-pteridinyl)(2-hydroxyethyl)amino)-2-propanol; TX-3301; 2,6-di-(2,2-dimethyl-1,3-dioxolan-4-yl)-methoxy-4,8-di-piperidinopyrimidopyrimidine; 2,6-bis-(2,3-dimethyoxypropoxy)-4,8-di-piperidinopyrimidopyrimidine; 2,6-bis[N,N-di(2-methoxy)ethyl]-4,6-di-piperidinopyrimidopyrimidine; and 2,6-bis(diethanolamino)-4 8-di-4-methoxybenzylaminopyrimidopyrimidine.

3. The method of claim 2, wherein said corticosteroid is prednisolone or prednisone and said tetra-substituted pyrimidopyrimidine is dipyridamole.

4. The method of claim 1, further comprising administering to said patient a third agent selected from the group consisting of antibiotics (penicillin, cephalosporin, tetracycline, oxytetracycline, chlortetracycline, metronidazole, chloramphenicol, streptomycin, neomycin, sulfonamides, phenolic compounds, quartemary ammonium compounds, minocycline, doxycycline); antiseptics (e.g., chlorhexidine); nonsteroidal antiinflammatories (e.g., flurbiprofen, carprofen, diclofenac, fenbufen, fenclozic acid, fenoprofen, flufenamic acid, ibuprofen, indomethacin, indoprofen, ketoprofen, lonazolac, loxoprofen, meclofenamic acid, mefanamic acid, naproxen, proprionic acids, salicylic acids, sulindac, tolmetin, meloxicam, oxicams, piroxicam, tenoxicam, etodolac, and oxaprozin); tranexamic acid, allantoin; epsilon-aminocaproic acid; lysozyme; dihydrocholesterol; and beta-glycyrrhetinic acid.

5. The method of claim 1, wherein said corticosteroid and tetra-substituted pyrimidopyrimidine are formulated in a single composition.

6. The method of claim 5, wherein said composition is formulated for oral administration.

7. The method of claim 5, wherein said composition is formulated for systemic administration.

8. The method of claim 1, wherein said corticosteroid and tetra-substituted pyrimidopyrimidine are administered simultaneously or within 14 days of each other.

9. The method of claim 1, wherein said corticosteroid or said tetra-substituted pyrimidopyrimidine is present in said composition in a low dosage.

10. A method for reducing serum C-reactive protein (CRP), IL-6, and/or IFN-γ levels in a patient in need thereof, said method comprising administering to said patient (i) a corticosteroid; and (ii) a tetra-substituted pyrimidopyrimidine, wherein said corticosteroid and tetra-substituted pyrimidopyrimidine are administered in amounts and for a duration that together are sufficient to reduce serum CRP, IL-6, and/or IFN-γ levels in said patient.

11. The method of claim 10, wherein said corticosteroid is prednisolone or prednisone and said tetra-substituted pyrimidopyrimidine is dipyridamole.

12. A method for treating periodontal disease, said method comprising administering to a patient (i) a corticosteroid; and (ii) a tetra-substituted pyrimidopyrimidine, wherein said corticosteroid and tetra-substituted pyripyrimyrimidine are administered in amounts and for a duration that together are sufficient to treat periodontal disease.

13. The method of claim 12, wherein the periodontal disease is periodontitis.

14. The method of claim 12, wherein the periodontal disease is gingivitis.

15. A device for administering drugs to the periodontal pockets of a patient having periodontal disease, said device comprising a corticosteroid and a tetra-substituted pyrimidopyrimidine capable of being released into the periodontal pockets of said patient.

16. The device of claim 15, wherein said tetra-substituted pyrimidopyrimidine is selected from the group consisting of 2,6-disubstituted 4,8-dibenzylaminopyrimido[5,4-d]pyrimidines; mopidamole; dipyridamole monoacetate; 1-((2,7-bis(2-methyl-4-morpholinyl)-6-phenyl-4-pteridinyl)(2-hydroxyethyl)amino)-2-propanol; TX-3301; 2,6-di-(2,2-dimethyl-1,3-dioxolan-4-yl)-methoxy-4,8-di-piperidinopyrimidopyrimidine; 2,6-bis-(2,3-dimethyoxypropoxy)-4,8-di-piperidinopyrimidopyrimidine; 2,6-bis[N,N-di(2-methoxy)ethyl]-4,6-di-piperidinopyrimidopyrimidine; and 2,6-bis(diethanolamino)-4,8-di-4-methoxybenzylaminopyrimidopyrimidine.

17. The device of claim 16, wherein said corticosteroid is prednisolone or prednisone and said tetra-substituted pyrimidopyrimidine is dipyridamole.

18. The device of claim 15, further comprising a third agent selected from the group consisting of antibiotics (penicillin, cephalosporin, tetracycline, oxytetracycline, chlortetracycline, metronidazole, chloramphenicol, streptomycin, neomycin, sulfonamides, phenolic compounds, quartemary ammonium compounds, minocycline, doxycycline); antiseptics (e.g., chlorhexidine); nonsteroidal antiinflammatories (e.g., flurbiprofen, carprofen, diclofenac, fenbufen, fenclozic acid, fenoprofen, flufenamic acid, ibuprofen, indomethacin, indoprofen, ketoprofen, lonazolac, loxoprofen, meclofenamic acid, mefanamic acid, naproxen, proprionic acids, salicylic acids, sulindac, tolmetin, meloxicam, oxicams, piroxicam, tenoxicam, etodolac, and oxaprozin); tranexamic acid, allantoin; epsilon-aminocaproic acid; lysozyme; dihydrocholesterol; and beta-glycyrrhetinic acid.

19. A kit comprising:
(i) a corticosteroid;
(ii) a tetra-substituted pyrimidopyrimidine; and
(iii) instructions for administering said corticosteroid and said tetra-substituted pyrimidopyrimidine to a patient having or at risk of having periodontal disease.

20. A kit comprising:
(i) a corticosteroid;
(ii) a tetra-substituted pyrimidopyrimidine; and
(iii) instructions for administering said corticosteroid and said tetra-substituted pyrimidopyrimidine to a patient having increased serum CRP, IL-6, and/or IFN-γ levels.

21. A kit comprising:
(i) a corticosteroid; and
(ii) instructions for administering said corticosteroid and a tetra-substituted pyrimidopyrimidine to a patient having or at risk of having periodontal disease.

22. A kit comprising:
(i) a corticosteroid; and
(ii) instructions for administering said corticosteroid and a tetra-substituted pyrimidopyrimidine to a patient having increased serum CRP, IL-6, and/or IFN-γ levels.

23. A kit comprising:
(i) a tetra-substituted pyrimidopyrimidine; and
(ii) instructions for administering said tetra-substituted pyrimidopyrimidine and a corticosteroid to a patient having or at risk of having periodontal disease.

24. A kit comprising:
(i) a tetra-substituted pyrimidopyrimidine; and
(ii) instructions for administering said tetra-substituted pyrimidopyrimidine and a corticosteroid to a patient having increased serum CRP, IL-6, and/or IFN-γ levels.

25. A kit comprising:
(i) a composition comprising a corticosteroid and a tetra-substituted pyrimidopyrimidine; and
(ii) instructions for administering said composition to a patient having or at risk of having periodontal disease.

26. A kit comprising:
(i) a composition comprising a corticosteroid and a tetra-substituted pyrimidopyrimidine; and
(ii) instructions for administering said composition to a patient having increased serum CRP, IL-6, and/or IFN-γ levels.

27. A kit comprising:
(i) a corticosteroid;
(ii) a tetra-substituted pyrimidopyrimidine; and
(iii) instructions for administering said corticosteroid and said tetra-substituted pyrimidopyrimidine to a patient having a disease or condition associated with increased serum CRP, IL-6, and/or IFN-γ levels.

28. A kit comprising:
(i) a corticosteroid; and
(ii) instructions for administering said corticosteroid and a tetra-substituted pyrimidopyrimidine to a patient having a disease or condition associated with increased serum CRP, IL-6, and/or IFN-γ levels.

29. A kit comprising:
(i) a tetra-substituted pyrimidopyrimidine; and
(ii) instructions for administering said tetra-substituted pyrimidopyrimidine and a corticosteroid to a patient having a disease or condition associated with increased serum CRP, IL-6, and/or IFN-γ levels.

30. A kit comprising:
(i) a composition comprising a corticosteroid and a tetra-substituted pyrimidopyrimidine; and
(ii) instructions for administering said tetra-substituted pyrimidopyrimidine and a corticosteroid to a patient having a disease or condition associated with increased serum CRP, IL-6, and/or IFN-γ levels.
